(19) United States
(12) Patent Application Publication
(10) Pub. No.: US 2010/0298748 A1
(43) Pub. Date: Nov. 25, 2010

(54) MOBILIZING NECK SUPPORT DEVICE

(75) Inventors: Mark Rosenfeld, Alingsas (SE); Stephan Mangold, Molndal (SE)

Correspondence Address: RENNER OTTO BOISSELLE & SKLAR, LLP, 1621 EUCLID AVENUE, NINETEENTH FLOOR, CLEVELAND, OH, 44115, US

(73) Assignee: CERVRITE AB, Alingsas (SE)

(21) Appl. No.: 12/863,221

(22) PCT Filed: Jan. 29, 2009
(86) PCT No.: PCT/SE09/50107

§ 371 Date: Jul. 16, 2010

(30) Foreign Application Priority Data
Jan. 29, 2008 (SE) 0800219-8

(51) Int. Cl. A61F 5/00

(52) U.S. Cl. 602/17

(57) ABSTRACT

The invention discloses a cervical collar which provides support, safety and stability to the neck of the wearer, and is arranged to prevent cervical flexion, extension and bilateral lateral flexion while allowing cervical rotation. The cervical collar comprises two parts; one back piece including a shoulder and back support piece attached to an adjustable neck piece and a chin support piece. The chin support piece can be lowered, raised and/or removed by the wearer of the collar in order to allow opening of the mouth sufficiently for eating or performing personal hygiene without unwanted extension of the neck.

### TECHNICAL FIELD

The present invention concerns an easily fitted and applied rigid cervical collar providing comfort, stability and immobility to the neck and head in the neutral position by preventing movement in all directions but allowing controlled rotation of the head and neck, only when prescribed and electronically monitored, to prevent further injury and enhance the management, healing and rehabilitation of neck disorders and traumatic neck injuries, such as whiplash injuries, with or without neurological signs and symptoms.

### BACKGROUND OF THE INVENTION

There is a consensus for the need of initial or prolonged immobilisation for a number of neck disorders including traumatic neck injuries. There is also evidence for the effectiveness of early neck movement (rotation) in a doctoral thesis (Rosenfeld, 2006, Whiplash-associated disorders from a physical therapy and health-economic perspective) which has shown that healing and rehabilitation of neck injuries in motor vehicle crashes were significantly improved, and costs for these injuries significantly reduced if patients early and regularly exercised the neck through repeated rotation exercises. This thesis confirms previous studies showing the benefits of early movement in the treatment of neck and other musculoskeletal injuries. On the other hand, initial immobilisation protects the neck from further damage when there is extensive injury to the neck or insufficient stability, while early initiated neck rotation addresses problems caused by damaged nerves, joints and soft tissue by mobilizing these structures, when tolerated, to prevent scar tissue from forming adhesions causing later dysfunction as well as by keeping neck muscles fit by preventing muscle atrophy, weakness and contraction. Movement also provides valuable stimulation of proprioception, essential for balance and coordination. Neck rotation also avoids the longitudinal stress on the neural axis caused by flexion, extension and lateral flexion. Mobilisation also reduces possible inhibition of blood flow due to compression. Animal studies have shown the negative effects of prolonged immobilization on joints and that cartilaginous, articular repair is significantly enhanced by movement and loading and is an important stimulus to joint regeneration processes. Early movement also addresses the issue of fear of movement and avoidance of activity, which together, can contribute to delayed recovery. Rotation exercises have thus become an accepted part of the treatment of acute whiplash injuries throughout the world. There is also a potential benefit to other conditions requiring initial stability followed by mobilisation through active movement.

There is an invention of a cervical collar, U.S. Pat. No. 2,474,200 from 1946 that provides stability for the head and allows neck rotation, but cannot be adjusted for the centre of rotation or rotation plane due to its rigid construction. The collar is consequently not “user-friendly” and is considerably more difficult to apply or remove for personal hygiene, eating or sleeping. The solid rings must be with difficulty drawn over the head. The jaw and neck support can be applied first after this procedure, and adjusted to the desired position by the patient. They provide support for the head and the chin but prevent the jaws from opening, causing obvious difficulties for eating and maintaining oral hygiene. It cannot be easily readjusted, because the nuts must be turned many turns by the patient in order to eat, nor easily returned to the correct, secured position due to the absence of suitable mechanical means. Hence, the patient can only maintain a liquid diet during treatment unless the collar is removed.

Another invention, WO 2006/020514 A2, has divided the ring to enable removal of the cervical collar without having to pull the shoulder parts or the ring over the head. However, the left and right sections for head support are difficult to put on or remove without help. The technical solution also prevents the patient from opening the mouth.

There are inventions where the support for the chin is adjustable as in U.S. Pat. No. 3,306,248, which describes how the chin cup prevents rotary motion of the chin. There is some adjustability to secure the chin cup in a fixed position on the base component. It cannot, however, be easily readjusted by the patient in order to eat, nor easily returned to the correct, secured position due to the absence of suitable mechanical means.

Another invention, US 2005/0010148, has a chin mastoid support where a cam member of the neck collar guides movement of the chin support relative to the support member. The neck collar allows stretching (flexion) and distraction (extension) of the neck.

The invention, WO 2006/069074, provides the collar with a chin mastoid piece for engaging and positioning the head, thereby correcting the head posture from the forward head position to an increasingly corrected (neutral) position. Neither of the inventions describes the problem or solution for the wearer to open the mouth in order to eat solid food or carry out oral hygiene.

An important aspect of any successful treatment is patient compliance. Monitoring usage is essential to evaluate treatment both in clinical trials and daily practice. Studies have shown that monitoring usage positively affects compliance, that is, the patient is more apt to follow the physician's instructions if monitored. Some collars have a built-in monitoring device such as US2003158015, the purpose of which is to provide feedback on resistance for neck flexor training. Monitoring is achieved by measuring pressure in the form of a mechanical pressure gauge connected to an air chamber. This provides feedback to the patient in order to maintain a specific pressure through muscle activation for a specified time to achieve a training effect. It does not, however, measure the number of times the patient carries out the exercise. It is important that the care provider can monitor usage electronically over a period of time to measure compliance, that is, that the patient actually carries out the prescribed exercise. It would be of great value to the patient to receive an alarm signal when it is time to exercise the head by turning the head in the prescribed manner as described in the doctoral thesis (Rosenfeld, 2006). It is necessary to both monitor and store information to provide data on compliance and usage. The information can then be transferred from the device to a computer to store and analyse this feedback information. There are cervical collars with integrated electrical circuitry including a small battery for electromagnetic field therapy, U.S. Pat. No. 6,024,691, but these are not intended for storing information for feedback purposes.

There is, at present, no orthopaedic, technical device or cervical collar that allows beneficial rotational movements of the head and neck while simultaneously providing stability and support that is so user-friendly that the user can independently put on or remove the collar. Neither are there cervical devices allowing beneficial, rotational movements of the head and neck while simultaneously providing stability and support that allow easily and sufficiently opening of the mouth during eating without unwanted extension of the neck which can cause potentially harmful stress to the injured structures of the neck. Neither are there cervical devices that can provide signals to the user to perform exercise with the head nor monitor and store information to provide data on compliance and usage.

### SUMMARY OF THE INVENTION

The present invention relates to an easily applicable cervical collar providing comfortable support, safety and stability to the injured neck by preventing cervical flexion, extension, bilateral lateral flexion and neck rotation but allowing controlled, neck rotation on demand. The centre of rotation and rotation plane can be adjusted for each user. The device, which consists of a semi-circular ring for rotation, can now be easily put on or removed by the patient without help. The chin support piece can be lowered to allow opening of the mouth and chewing, thereby allowing solid food intake and easing the performance of oral hygiene. The device incorporates an electronic unit, to give an alarm signal to the user to perform the described exercises, monitors and stores information on how the user has utilized the device during a certain period of time in order to provide information on compliance. This is useful for both research and clinical purposes and positively influences compliance. Noncompliance, or not following physician's prescribed treatment, is a great problem in medicine leading to delayed healing, cure and rehabilitation.

This new, user-friendly, neck stabilizing and mobilizing device provides protection, safety, and comfort to patients requiring neck stability and therapeutic and rehabilitating rotational movement of the neck by adjusting the rotation centre and rotation plane. It also provides the possibility to move the jaw when necessary to allow eating and oral hygiene while compliance is increased through built-in monitoring and initiating movement electronically using a built-in alarm. This provides totally new possibilities for treating both acute, subacute and chronic neck disorders, unachievable today with the present assortment of cervical collars and braces. This unique invention combines stability and mobility without interfering with activities of daily living. The user-friendly, open design permits ease of application and removal as well as comfort and hygiene. These unique features of user-friendliness, stability, mobility, increased compliance through an alarm signal and monitoring (providing feedback to both user and prescriber), a removable and adjustable jaw support for eating and oral hygiene are all combined to provide the essentials for maximum healing in one device easily adjustable for each user.

In particular, the invention relates to a cervical collar providing support, safety and stability to the neck of the wearer, arranged to prevent cervical flexion, extension and bilateral lateral flexion while allowing cervical rotation, said cervical collar comprising two parts; one back piece including a shoulder and back support piece attached to an adjustable neck piece, and a chin support piece; said adjustable neck piece further comprising a lower neck support piece and an upper neck support piece, said lower neck support piece being attached to the shoulder and back support piece in an adjustable manner, and being connected to an upper neck support piece by a rotational means, said rotational means allowing circular horizontal rotation between the neck support piece 206 in relation to the lower neck support piece; and wherein said chin support piece is connected to said adjustable neck piece by a release and locking means enabling the chin support piece to be tilted, lowered, raised and/or removed by the wearer in order to allow opening of the mouth sufficiently for eating or performing personal oral hygiene without unwanted extension of the neck, the attachment between the shoulder and back support and the lower neck support piece is provided by at least three supportive connecting means which preferably are adjustable in the lengthwise direction and angular position with regards to the shoulder and back support in relation to the rotational means.

In another embodiment of the invention the supportive connecting means are rigging screws provided with ball-and-socket joints at either ends, said balls being fitted into gripping sockets provided on the shoulder and back support and the lower part of the rotational means.

In another embodiment of the invention the circular horizontal rotation of the lower neck support piece in relation to the neck support piece is permitted by a rotational means comprising two semi-circular rings which are connected to each other in a way which substantially reduces friction.

In another embodiment of the invention the lower semi-circular ring is connected to an upper semi-circular ring by means of an I-beam profile functioning as a sliding bearing.

In another embodiment of the invention the I-beam profile is made from low friction plastic like PTFE (PolyTetraFlourEtylen).

In a further embodiment of the invention release and locking means are present on each side of the collar, said means comprising actuators that when engaged by the user of the collar enables the chin support piece to be lowered, raised and/or removed.

In another embodiment of the invention engagement of the actuators shifts the release and locking means from a locked position to an unlocked position enabling the chin support piece to be lowered, removed and/or adjusted in the lengthwise direction, and when said chin support piece is reinserted and/or raised by the hand of the user the release and locking means returns to the locked position at which the chin support piece 203 provides the required support for the chin and head.

In another embodiment of the invention the release and locking means further comprises an adjustment means enabling the chin support piece to be tilted in relation to the neck support piece.

In another embodiment of the invention the adjustment means is an angular adjustment means enabling an incremental angular adjustment of the chin support piece of less than 5°, more preferably of less than 3° and most preferably of less than 2°.

In another embodiment of the invention the angular adjustment means is a cogged metal ring.

In another embodiment of the invention the circular horizontal rotation between the lower neck support piece in relation to the neck support piece is restricted with respect to each other by adjustable regulating means.

In another embodiment of the invention the adjustable regulating means are stop lugs.

In another embodiment of the invention the circular horizontal rotation of the lower neck support piece in relation to the neck support piece is adjustable from a few degrees to the maximum.

In another embodiment of the invention the circular horizontal rotation of the lower neck support piece 205 in relation to the neck support piece with respect to each other is completely prevented.

A further embodiment of the invention comprises an electronic monitoring device arranged to register rotational movements of the cervical collar.

In another embodiment of the invention the electronic monitoring device comprises means for registering, monitoring and storing information about rotational movements preformed by the wearer of the cervical collar.

In another embodiment of the invention the means are micro circuit breakers or magnetic reed switches, a magnet and a micro computer.

In another embodiment of the invention the stored information in the electronic monitoring device can be read out by a computer.

In another embodiment of the invention the stored information in the electronic monitoring device can be read out to the user's cell phone and/or personal digital assistant (PDA).

In another embodiment of the invention the electronic monitoring device has a built in alarm.

In another embodiment of the invention the intervals between alarms can be programmed in the electronic monitoring device.

The invention further relates to a method for treating neck injuries, including whiplash injuries, whereby the cervical collar according the invention is applied around the damaged neck, providing support, safety and stability to prevent cervical flexion, extension and bilateral lateral flexion of the neck while allowing cervical rotation.

Additional features and advantages of the invention will appear more clearly from the following detailed description of some preferred embodiments of the invention, which is given by way of non-limiting example only and with reference to the accompanying drawings.

### BRIEF DESCRIPTION OF THE DRAWINGS

FIG. 1 illustrates how the cervical spine and head (oval ring) rotate in relation to the device's circular form at the spinal axis of rotation.

FIG. 2a illustrates the device (lateral view) on the patient.

FIG. 2b illustrates the device from the anterior position.

FIG. 3 illustrates the parts for allowing circular horizontal rotation.

FIG. 4a shows a cross-section of the semi-circular metal ring profile.

FIG. 4b shows a cross-section with an integrated I-profile.

FIG. 5 shows a detailed drawing of the parts of the release and locking device shown from the inside of the right side for the chin support piece of the device.

FIG. 6 shows the right side release and locking device for the chin support piece in the locked position.

FIG. 7 shows the right side release and locking device for the chin support piece in the lower position.

FIG. 8 shows the semi-circular ring in a posterior view with the permanent and adjustable lugs.

FIG. 9 shows the inside of the semi-circular ring with the electronic monitoring device, magnetic sensors, and magnet.

FIG. 10 illustrates how, in a user-friendly way, the device can be put on.

### DETAILED DESCRIPTION OF PREFERRED EMBODIMENTS

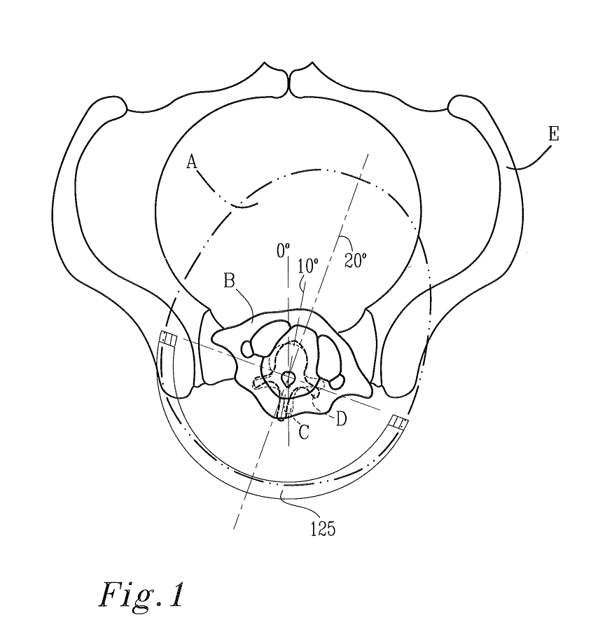

The following is a description of an embodiment of the invention, a device in the form of a new, user-friendly cervical collar for stabilization and immobilization of the neck while allowing rotation of the head and neck. FIG. 1 shows how the upper segments of the cervical spine (B, C, and D) move in connection with right head and neck rotation with the head (A) shown as an oval ring. The axis of rotation is located in the centre of the atlas (B). When there is rotation of the head and neck for 20 degrees, a few degrees of rotation occurs between the occiput and the atlas at the atlanto-occipitial joint. The remaining rotation up to 20 degrees occurs between B and C segments. The contours of the pelvis (E) are seen at the bottom of the drawing. The rotational plane and center varies somewhat along with anatomical variations in non neck-injured individuals and even more so in neck-injured individuals. It is therefore essential to be able to adjust the rotational means (125) of the cervical collar to each patient with regard to both anatomical structural variations and consequently variations in the rotational plane and center. The axis of rotation of the collar should lie within the cervical spine's axis of rotation. This entails that the actual plane of rotation of the collar must be circular in cross-section to allow head and neck rotation. However, the upper section of the collar must be formed so that the head (mandible and occiput) rest firmly supported while the lower section rests stably against the clavicle and shoulders, thus stabilising the head and neck and restricting cervical flexion, extension, and lateral flexion but allowing rotation when prescribed or needed. This is not possible in existing patented designs.

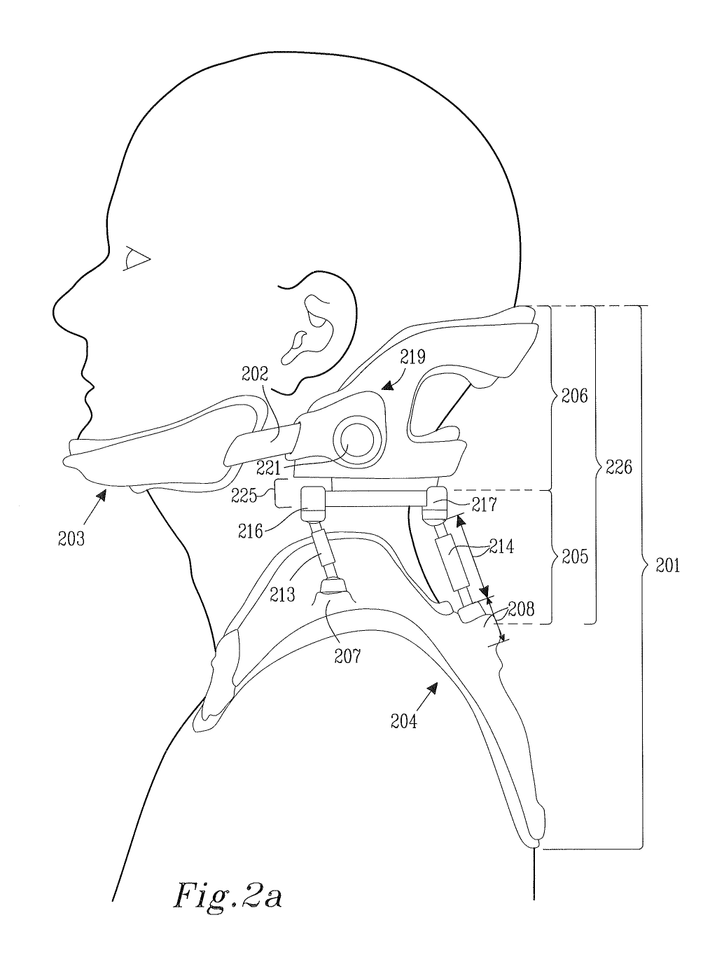

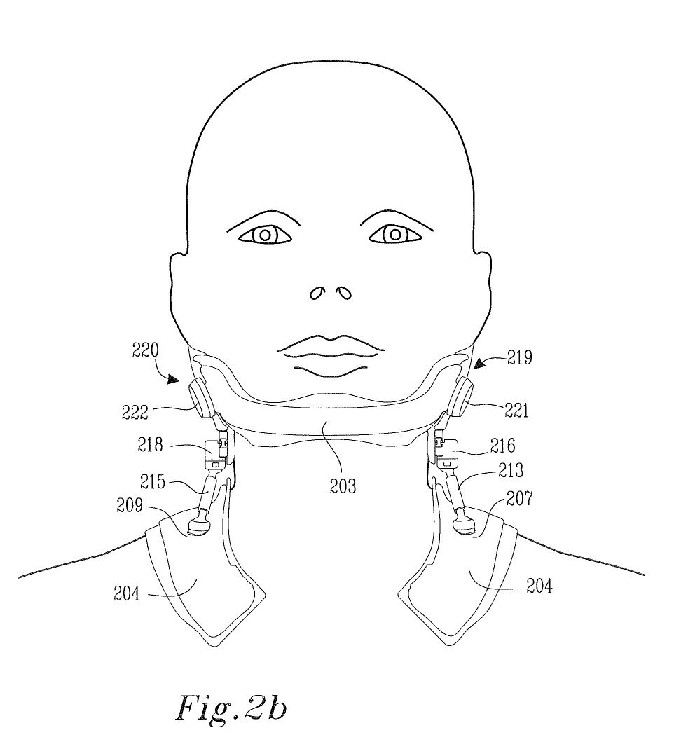

FIG. 2a is a side view of how the cervical collar is fitted on the patient and FIG. 2b shows a frontal view. The cervical collar comprises two pieces; one back piece (201) including a shoulder and back support piece (204) attached to an adjustable neck piece (226), and a chin support piece (203). Optionally a front piece (not shown) can be fitted to the shoulder and neck support piece on the wearer's chest for additional support. The front piece can be removed and refastened to the back piece (204) in the manner of snap locks or touch fasteners such as Velcro. The front piece and back piece (204), when joined together provide a base providing stability for the head as the back piece (204) reaches about half way down the middle of the user's back, and the front piece reaches down the middle to the end of the user's breast bone (sternum).

The adjustable neck piece (226) is the most complex part of the cervical collar comprising two parts: a lower neck support piece (205) and an upper neck support piece (206). Said lower neck support piece (205) is attached to the shoulder and back support (204) in an adjustable manner and is connected to an upper neck support piece (206) by a rotational means (225) which consists of a semi-circular ring enabling easy removal and application of the circular collar without having to pull it over the head, said rotational means allowing circular horizontal rotation between the neck support piece (206) in relation to the lower neck support piece (205). By using a semi-circular ring to provide rotation entails reduction of the radius of the ring in comparison to a rigid ring. This makes the cervical collar smaller and more user-friendly.

The attachment between the shoulder and back support (204) and the lower neck support piece (205) is provided by at least three supportive connecting means (213-215) which preferably are adjustable in the lengthwise direction. In this embodiment of the invention these connection means are comprised of adjustable rigging screws (213, 214, 215), which, at their lower ends, are fastened to the shoulder and back support (204) and in their upper ends to the lower part of the rotational means (225) of the neck support piece (206). Two of the supportive connection means (213 and 215) rest on each shoulder, and at least one supportive connection means (214) rests on the back of the user. Each supportive connection device (213-215) can be adjusted to the appropriate length and angle to assure a proper fit for the centre of rotation and rotational plane of the patient's neck as follows. The lower part of the supportive connection means (213-215) are provided with ball-and-socket joints. The balls of the ball-and-socket joint are fitted into gripping socket foundations (207-209) fixed onto the shoulder and back support (204). If the gripping socket (207-209) is tightened the supportive connection means is placed in a fixed angular position. By slightly loosening the gripping socket (207-209), the angle of the supportive connection means can change angle with regard to the shoulder and back support (204). The upper parts of the supportive connecting means (213-215) are fitted into similar gripping sockets (216-218) as said gripping sockets (207-209) at the lower ends. The upper gripping sockets (216-218) are connected to the lower part of the rotational means (225) on the neck support piece (206). In this embodiment of the invention the rigging screws (213-215) can be shortened or lengthened by turning their outer part. In this way it is possible to adjust the distance and angle between the rotational means (225) and the shoulder and back support (204) by means of loosening and tightening the gripping sockets (207-209, 216-218) and lengthening or shortening the rigging screws (213-215) assuring the correct centre of rotation and rotational plane of the patient's neck in rotation. FIG. 1 shows how the centre of rotation for the rotational means (125) shown as a circular ring has been adjusted to coincide with the spinal axis of rotation. Normally the physician or physical therapist makes these adjustments.

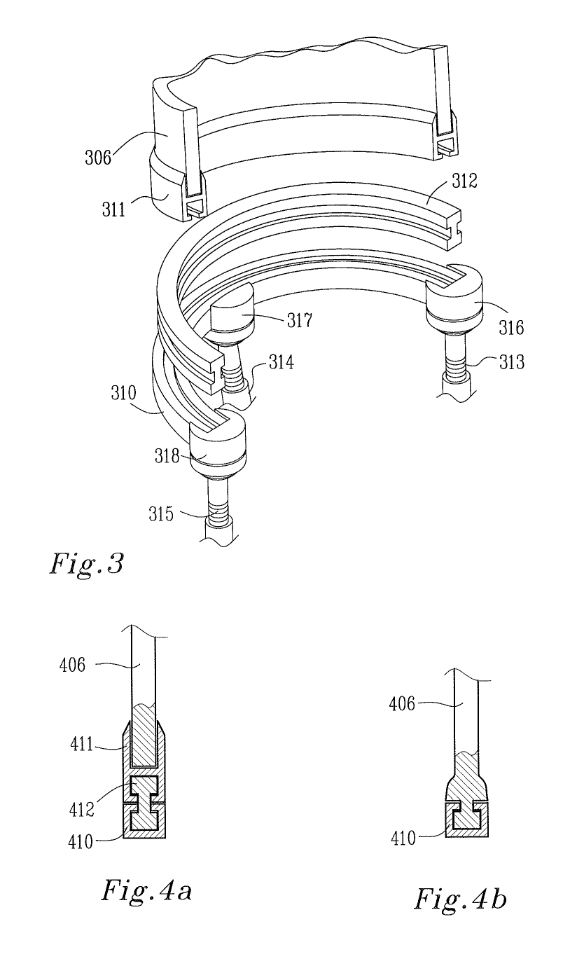

In FIG. 3 it can be seen how circular horizontal rotation between the shoulder and back support (204) in relation to the neck support piece (206) is permitted by a rotational means (225) which in this embodiment comprises two semi-circular rings (310 and 311) which are connected to each other in a way which substantially reduces friction.

The lower semi-circular ring (310) is connected to an upper semi-circular ring (311) by means of an I-beam profile (312). FIG. 4a shows the cross-section of the lower (410) and upper (411) semi-circular rings fitted with the I-profile (412). The semi-circular rings (410, 411) can be of metal such as aluminium and the I-beam profile (412) of any suitable low friction polymer such as for example PTFE (PolyTetraFlourEtylen). The profiles of the semi-circular ring and the I-beam profile are constructed so that rotation occurs only between the semi-circular ring and the I-beam profile to reduce friction. For a pulling force separating the two semi-circular rings from each other friction will occur at the inside part of the I-beam. If the two semi-circular rings are pressed together friction will occur between the inside parts of the semi-circular rings and the top of the I-beam. There is, thus, no friction between the metals of the semi circular rings (410 and 411) because they do not come in contact with each other due to the height of the I-beam profile.

The upper gripping sockets (316-318) which are connected to the connection means (213-215) are placed on the lower semi-circular ring (310). The I-beam profile (312) is held firmly in position by the upper gripping sockets on each side (316, 318) of the lower semi-circular ring (310). A neck support piece (306, 406) is fitted onto and fastened by for example glue to the upper semi-circular ring (311, 411).

In one embodiment of the invention the neck support piece (406) is totally integrated with the semi-circular ring (411) and the I-beam profile (412), are all made from the same material, such as for example plastic. The lower part of the I-beam fits into the rail of the semi-circular ring (410) shown as a cross section in FIG. 4b.

On each side of the upper neck support piece (206) there is a release and locking device (219, 220) to fasten the chin support piece (203) which can be lowered, raised or removed by simply engaging actuators (221, 222) on each side. This movement can be achieved by the wearer of the collar.

A person skilled in the art realizes that the release and locking device (219, left side and 220, right side) for the chin support piece (203) can be implemented in many ways. However, the main principle is that the user unlocks the chin support piece by pressing or turning actuators (221, 222) of the lock so that the chin support piece (203) can be lowered. The chin support piece (203) can also be fully removed from that position. It can then be reinserted to one of several positions (depending on the desired length of the chin support piece (203)), and by a simple hand movement thereafter be raised to the stable locked position.

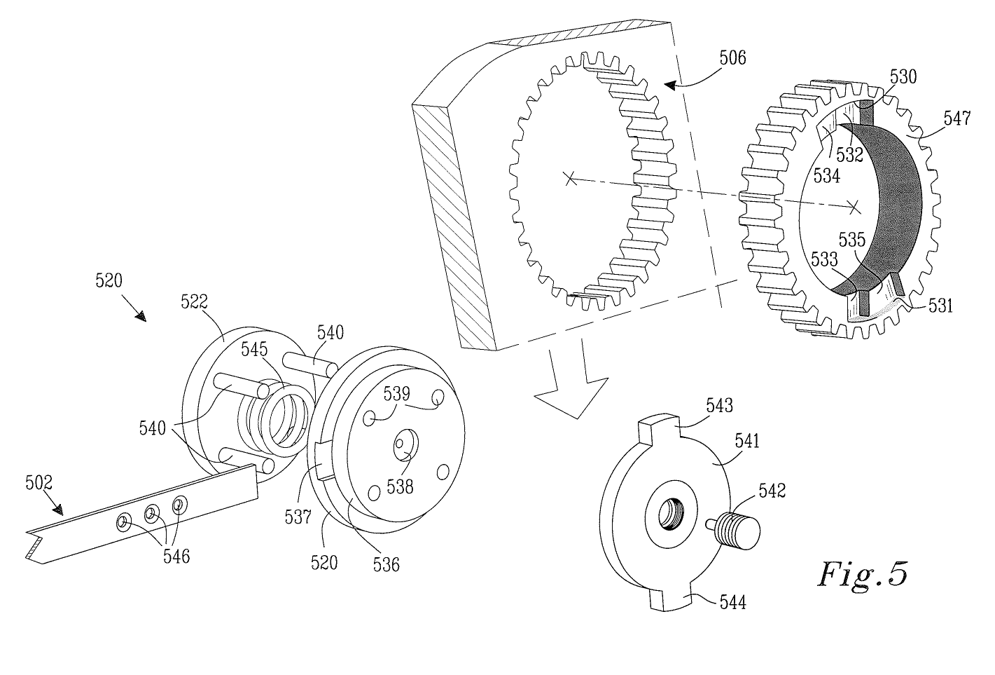

The release and locking device (219, 220) will now be described in detail with reference to FIG. 5 which shows an exploded view of one embodiment of the release and locking device (220) on the right side. The locking device, starting from the outside towards the cheek of the wearer, comprises essentially four parts; an actuator (522), a chin support holding device (520), an angular adjustment means (547), and an end piece (541). When connected together the release and locking device (219, 220) extends through a through-hole on the neck support piece (506). The actuator (522) which faces the outside of the neck support piece (506) and which is easily reached by the user, may consist of a flat button or knob having two or more connecting pins (540) surrounding a wave-spring (545) protruding from the inside face of the actuator (522) towards the wearer. The connecting pins (540) are arranged to pass through two or more through-holes (539) provided in the chin support holding device (520). The chin support holding device (520) is the part of the locking device that receives and holds a metal guide (502) of the chin support piece (203). One side of the chin support holding device (520) (the side facing the wearer) is cut out into a circular shaped insert (536) so as to fit into a round through-hole of the angular adjustment means (547) which is arranged in the neck support piece (506).

The chin support holding device (520) is provided with a guided, rectangular hole (537) into which the chin support piece metal guide (502) is received, and a hole (538) in the centre of the circular shaped insert (536) for locking the chin support piece metal guide (502) in the appropriate position with the means of a metal pin (542). The connecting pins (540) of the push button (522) pass through the through holes (539) of the chin support holding device (520), through the insert (536), said insert (536) fitting into the through-hole of the angular adjustment means (547) in the neck support piece (506) and the ends of the connecting pins (540) connect to the end piece (541) placed on the other side of the through-hole of the angular adjustment means (547) in the neck support piece (506). In this embodiment the connections consist of four pins (540) passing through the holes (539) of the chin support holding device with insert (520 and 536), and are connected (glued or welded) to the end piece (541) with a metal pin (542) in the centre.

The precise angle and height of the chin support piece (203) as required by each patient should in conjunction with the neck support piece (206) be adjusted during the initial fitting of the cervical collar which is performed by a physician or physical therapist. The angular position of the chin support piece (203) can be adjusted by changing the relative position of the angular adjustment means (547) to the neck support piece (506). In this embodiment of the invention the angular adjustment means is a cogged metal ring (547), and the angle of the chin support piece (203) is changed by pressing out the cogged metal ring (547) from the neck support piece (506), rotating the cogged metal ring (547) the number of cogs required to achieve the correct angle, and pressing the cogged metal ring (547) back into the neck support piece (506) again. The number of cogs present on the cogged metal ring (547) determines the minimum degree by which the angular position of the chin support piece (203) can be adjusted. Preferably the angular adjustment means should enable a stepwise incremental adjustment angle of <5°, more preferably of <3° and most preferably of <2° in order to fine-tune the correct angle of the chin support piece (203) in relation to the neck support piece (506) according to the physical requirements of the wearer. However, the person skilled in the art realizes that the angular adjustment of the chin support piece (203) can be accomplished by other means.

The metal guide (502) of the chin support piece (203) which comprises several holes (546), will when inserted into the rectangular hole (537) of the support holding device (520), determine the appropriate length of the chin support piece for each patient. How close the chin support piece (203) should be to the chin is recommended by the physician but the user has full control of how tight the chin support piece (203) shall be to allow for maximum comfort.

The height of the chin support piece (203) i.e. the level at which the user should keep his chin, is thereafter adjusted by loosening a screw (not shown) under the chin support piece (203) adjusting the angle between a metal guide (502) and chin support piece (203), thereafter tightening the screw again. This height, length and angular adjustment of the chin support piece (203) is to ensure a proper fit of said piece to the users chin and also to ensure that a correct position of the rotational plane and rotational centre is achieved.

One of the advantages of the cervical collar of the present invention is that the chin support piece is easily lowered, raised and/or removed by the user. This is accomplished as follows: the angular adjustment means (547) present in the neck support piece (506) is provided with a circular through-hole, the inside wall of which is provided with two or more recesses (530, 531) placed opposite each other on either side of the through-hole, each with two landings at different depths, one for the locked position (532, 533) and one for the lowered chin support position (534, 535).

The end piece (541) positioned on the other side of the through-hole of the angular adjustment means (547) in the neck support piece (506) has two “earlike” extensions on opposite sides (543, 544), which fit into the recesses (530, 531) of the through-hole of the angular adjustment means (547) in the neck support piece (506). The wave-spring (545), located between the push button (522) and the chin support holding device (520), ensures, when pushing the chin support piece upwards, that the pin (542) automatically enters the locking device through one of the holes (546) on the chin support piece metal guide (502). The connecting pins (540) also transfer the turning force of the chin support piece metal guide (502) to the end piece (541) when it is lowered by the chin or by the hand of the user.

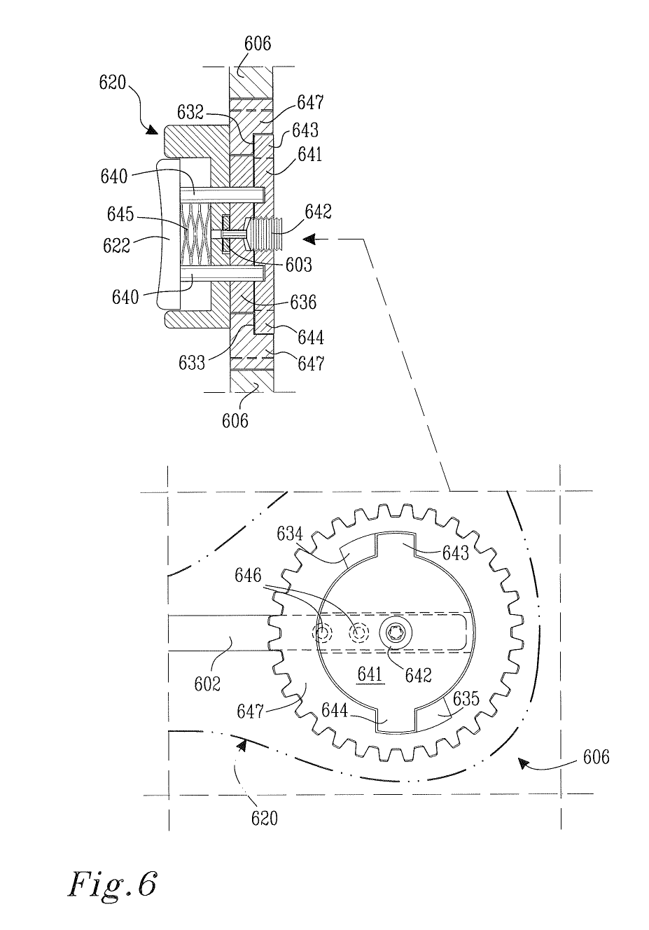

In locked position as illustrated in FIG. 6, the earlike extensions (643, 644) are on the lowest landings (632, 633) and the metal pin (642) goes through one of the holes (646) in the chin support piece metal part (602). The release and locking device on the opposite side of the neck support piece (506) is similar although mirrored. Hence the chin support piece metal guide (602) is locked and prevented from moving in and out by the metal pin (642) and from lowering due to the “earlike” extensions (643 and 644) in the lower landings (632 and 633) of the recesses.

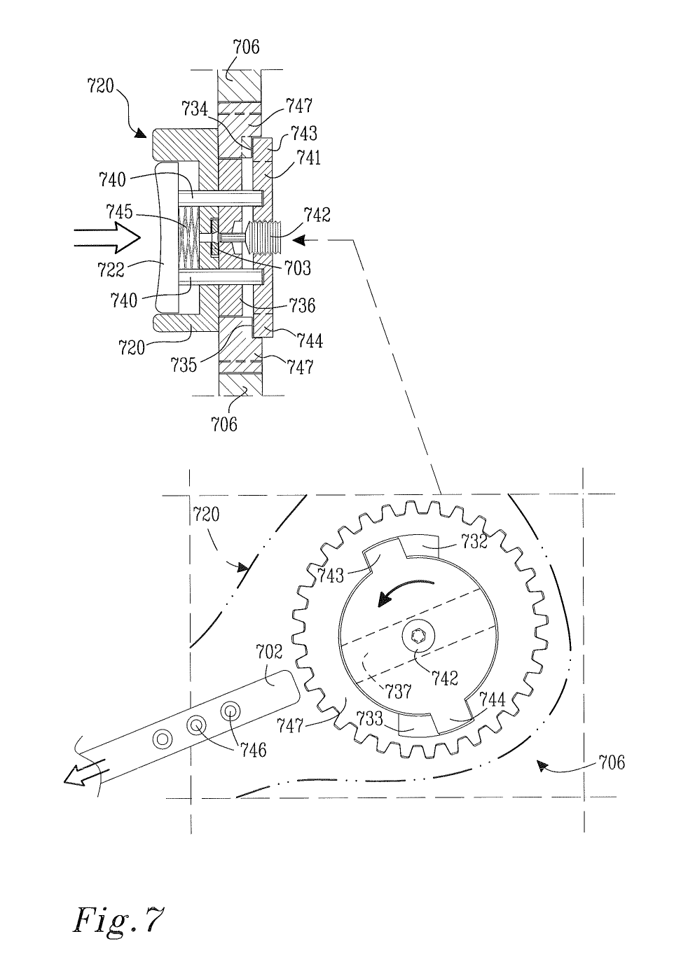

FIG. 7 shows that when the user wants to lower the chin support piece (203) the user presses the actuators (721, 722) on each side of the neck support piece (706), The wave-spring (745) is compressed between the actuator (722) and the holding device (720) whereby the connecting means (740) push the end piece (741) towards the cheek of the wearer and pulls the metal pin (742) out of the hole (746) in the chin support piece metal guide (702). Now the chin support piece (203) is in the unlocked position and can be lowered as shown in FIG. 7, whereby the earlike extensions (743, 744) move to the higher landings (734, 735). When the ear-like extensions (743, 744) are positioned on the higher landings (734, 735) the tip of the metal pin (742) just barely holds the chin support piece metal guide (702) in position. This can be precisely adjusted so that the metal pin (742) can be turned and screwed in the right position. By simply pushing the chin support piece upwards it will automatically return to the stable locked position because of the action of the wave-spring (745). In the lowered position the pin (742) reaches only slightly into one of the holes (746) in the metal guide (702) of the chin support piece (203). The pin (742) still retains a holding position for the chin support piece (203) but by pulling the chin support piece outwards it can easily be removed. The chin support piece can again be inserted into the rectangular holes (737) in order to attain the correct position that can be felt when the pin (742) snaps from one hole (746) to the next due to the action of the wave-spring (745). When the correct position is reached the chin support piece (203) is pushed upwards and enters the locked position.

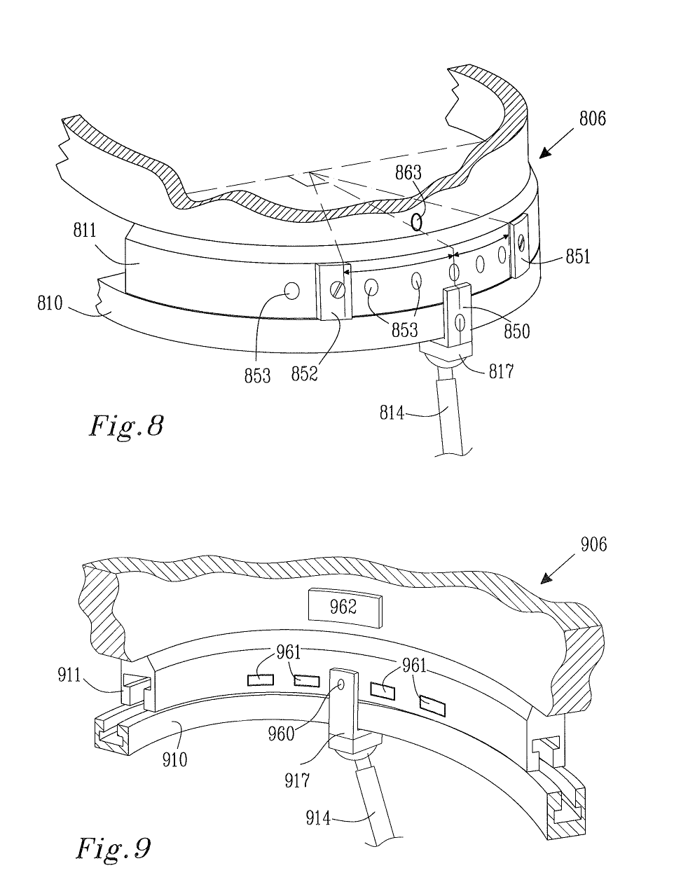

The circular horizontal rotation between the lower neck support piece (205) in relation to the neck support piece (206) can be restricted with respect to each other by adjustable regulating means. Locking can be necessary in the early stages of the healing process or in the case of instability due to fracture or ligamentous insufficiency. In FIG. 8 it can be seen that in the back of the lower semi-circular ring (810) one of the gripping sockets (216-218), preferably one in the back (817) of the connecting means (814) also serves as a stop lug (850) as shown in FIG. 8. The upper semi-circular ring (811) is equipped with adjustable stop lugs (851, 852), so that varying degrees of rotation can be allowed depending on the degree of healing, tissue and patient tolerance. There is, thus a possibility to determine the degree of rotation from a few degrees to maximum degrees by adjusting and locking the stop lugs in the predrilled holes (853) in the semi-circular ring (811). In this embodiment the stop lugs (851, 852) are put in place with a screw for each stop lug, and screwed in to one of the holes (853) which is threaded. The collar can also be locked to completely prevent rotation by setting the stop lugs (851, 852) close to the stop lug (850) of the gripping socket (817).

To register compliance by monitoring the number of rotation exercises performed, the device is equipped with an electronic monitoring device. The stop lugs (851, 852) are equipped with micro current circuit breakers, or as shown in FIG. 9, magnetic reed switches (961) on the inside of the upper semi-circular ring (911) and a magnet (960) on the inside of the gripping socket (917). The micro current circuit breakers or magnetic reed switches are connected to a low power, single chip computer such as for example the PIC12C50, and a real time clock such as for example the DS1302, powered by a single cell battery. The computer, battery and accessories are mounted in a small electronic unit (962) fastened, for example, inside the lower rear part of the neck support piece (906). At a follow-up appointment, data can be gathered from the electronic monitoring device via the connector (863), which is connected to the physician's computer, for example, via a USB port. It is then possible to see how the patient has utilized the cervical collar by seeing, for example, how often and when the head has been rotated throughout the healing process. The digital transfer between the electronic monitoring device and the computer can be wireless made, optically with infrared waves (IR) or with radio waves (Bluetooth). If a wireless transmission is used, such as Bluetooth, the information can be transferred to the user's cell phone, personal digital assistant (PDA) or computer in order to notify the user how many rotations have been made during the day. With this information the user can increase the number of rotations if the user has not reach the prescribed number for the day. The electronic monitoring device can also send an alarm signal to the user's cell phone, personal digital assistant (PDA) or computer to remind them to perform the rotation exercise. The alarm signal can also be installed in the software of the user's cell phone, personal digital assistant (PDA) or computer. The electronic unit (962) can also be equipped with a sounding alarm, such as a small loudspeaker, producing an alarming signal, for example hourly, when the user is to perform the rotation exercise. The physician can pre-program or reprogram the intervals between alarms.

Now follows a description in order to describe how the orthopaedic technician or person the like will fit and adjust the cervical collar to a patient. First a right in size, shoulder and back support piece (204) is chosen for the patient. It can be chosen from four different sizes like small, medium, large and extra large. The six gripping sockets (207-209, 216-218) of the supportive connecting means (213-215) are slightly loosened and the adjustable neck piece (226) is folded backwards. The back support piece (204) is put on the patient and adjusted, if necessary, by slightly bending the angle between shoulder and back. The part are now comfortably fitted to the patient's torso in order to give stability to the neck support piece (206) The technician adjusts the connection supports (213-215) to the proper length to provide the correct plane and centre of rotation for the individual head movement of the patient and that the neck of the patient comfortably fit into the neck support piece (206). The six ball-and-socket joints which for the moment are slightly loosened are now possible to adjust into an angle which makes it possible to find the right position for the rotational means (125) with respect to the correct plane and centre of rotation (see FIG. 1). When the correct position is found the six gripping sockets (207-209, 216-218) of the supports are tightened.

The technician makes sure that the patient can turn the head comfortably, readjusting if necessary. The technician chooses from four different sizes such as small, medium, large and extra large as a suitable chin support piece (203) for the patient and selects the proper hole (546) by inserting it into the release and locking device (219, 220), so that the head maintains good stability between the chin support device (203) and the neck support piece (206). If the angle and or height between the chin support piece (203) and the neck support piece (206) is not optimal the angle can be readjusted by pressing out the cogged metal ring (547) on both sides and pressing them in again in the neck support piece (206) as described above. But first, the screw under the chin support piece is loosened so that the angle between the chin support piece (203) and the metal part (202) can be readjusted for optimal fitting of the chin support piece (203) to the user's chin. After this adjustment the screw is tightened again. According to doctors prescription the stop lugs (851, 852) are screwed in place to allow the prescribed rotation of the head. The technician connects the electronic monitoring device to the connection socket (863) of the cervical collar to the computer and resets the counters and, if needed, programs the alarm in the electronic unit. If the patient has a cell phone with Bluetooth or the like, the technician downloads a software program to the cell phone so that the patient can receive the alarm signal and also check how he or she has used the cervical collar.

At a return visit to the technician or doctor, stored content of information can be read out from the memory of the electronic monitoring device into a computer and hence determine how the patient has used the cervical collar during the treatment period.

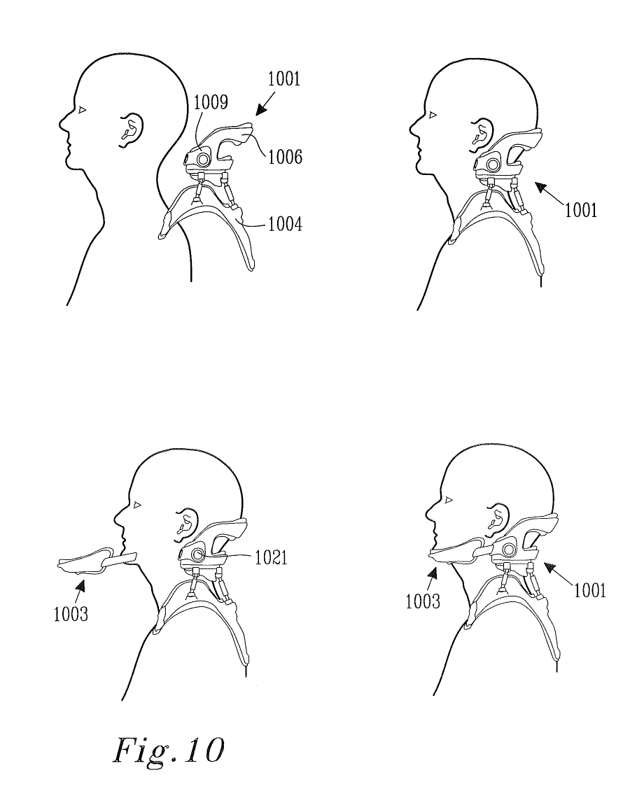

It is very important that the cervical collar is user-friendly and comfortable to obtain patient acceptance. FIG. 10 illustrates how the patient can apply the device, once fitted by a technician as described above, without help. By using a semi-circular ring as the rotational means makes the radius of the ring smaller in comparison to a rigid closed ring. This will in turn make the cervical collar smaller allowing removal without having to pull the cervical collar over the head. The patient begins with the back support piece (1001) with the adjusted neck support device (1006) and puts it on the shoulders and back. The chin support piece (1003) is then slid into the release and locking device (1019, 1020) and locked into position by pushing the chin support piece upwards.

When the patient wants to eat, he or she simply presses the buttons (1021, 1022) on the sides of the neck support piece, and the chin support piece (1003) can be lowered into position so that the patient can chew unhindered. Lowering is achieved by opening the mouth and thereby pushing the chin support device (1003) downward. After eating, the patient simply moves the chin support piece (1003) upwards by hand to the locked position. To remove the cervical collar the patient presses the buttons (1021, 1022) so that the chin support piece is moved to the lower position where it can be removed by pulling it forward. The patient then takes off the back support piece (1001) with the adjustable, but fixed, neck support piece (1006). For optimal function and comfort the inside of the cervical collar is form-fitted with a comfortable material such as semi-rigid foam. It can be removed, washed and put back in place. In this way, another patient can use the cervical collar when treatment has been successfully completed for the previous patient. Thus, the cervical collar is very user-friendly, cost-effective and comfortable and can be used even while eating or brushing teeth.

The device is thus not limited to the previously described embodiments but is designed for the features of combining user friendliness, comfort and compliance in the treatment of neck injuries such as whiplash injuries.

1. A cervical collar providing support, safety and stability to the neck of a wearer, arranged to prevent cervical flexion, extension and bilateral lateral flexion while allowing cervical rotation, said cervical collar comprising two parts;
a back piece including a shoulder and back support piece attached to an adjustable neck piece, and a chin support piece; said adjustable neck piece further comprising a lower neck support piece and an upper neck support piece, said lower neck support piece being attached to the shoulder and back support piece in an adjustable manner, and being connected to an upper neck support piece by a rotational means, said rotational means allowing circular horizontal rotation between the neck support piece in relation to the lower neck support piece;
said chin support piece is connected to said adjustable neck piece by a release and locking means enabling the chin support piece to be tilted, lowered, raised and/or removed by the wearer in order to allow opening of the mouth sufficiently for eating or performing personal oral hygiene without unwanted extension of the neck, wherein
the attachment between the shoulder and back support and the lower neck support piece is provided by supportive connecting means which preferably are adjustable in the lengthwise direction and angular position with regards to the shoulder and back support in relation to the rotational means.

2. A cervical collar according to claim 1, wherein the supportive connecting means are rigging screws provided with ball-and-socket joints at either ends, said balls being fitted into gripping sockets provided on the shoulder and back support and the lower part of the rotational means.

3. A cervical collar according to claim 1, wherein circular horizontal rotation of the lower neck support piece in relation to the neck support piece is permitted by a rotational means comprising two semi-circular rings which are connected to each other in a way which substantially reduces friction.

4. A cervical collar according to claim 3, wherein the lower semi-circular ring is connected to an upper semi-circular ring by means of an I-beam profile functioning as a sliding bearing.

5. (canceled)

6. A cervical collar according to claim 1, wherein release and locking means are present on each side of the collar, said means comprising actuators that when engaged by the user of the collar enables the chin support piece to be lowered, raised and/or removed.

7. (canceled)

8. A cervical collar according to claim 1, wherein the release and locking means further comprises an adjustment means enabling the chin support piece to be tilted in relation to the neck support piece.

9. A cervical collar according to claim 8, wherein the adjustment means is an angular adjustment means enabling an incremental angular adjustment of the chin support piece of less than 5°, more preferably of less than 3° and most preferably of less than 2°.

10. (canceled)

11. A cervical collar according to claim 1, wherein the circular horizontal rotation between the lower neck support piece in relation to the neck support piece is restricted with respect to each other by adjustable regulating means.

12. (canceled)

13. A cervical collar according to claim 1, wherein the circular horizontal rotation of the lower neck support piece in relation to the neck support piece is adjustable from a few degrees to the maximum.

14. A cervical collar according to claim 1, wherein the circular horizontal rotation of the lower neck support piece in relation to the neck support piece with respect to each other is completely prevented.

15. A cervical collar according to claim 1, wherein the cervical collar it further comprises an electronic monitoring device arranged to register rotational movements of the cervical collar.

16. A cervical collar according to claim 15, wherein the electronic monitoring device comprises means for registering, monitoring and storing information about rotational movements performed by the wearer of the cervical collar.

17. A cervical collar according to claim 16, wherein the means for registering, monitoring and storing, information are micro circuit breakers or magnetic reed switches, a magnet and a micro computer.

18. A cervical collar according to claim 15, wherein the stored information in the electronic monitoring device can be read out by a computer.

19. A cervical collar according to claim 15, wherein the stored information in the electronic monitoring device can be read out to the user's cell phone and/or personal digital assistant (PDA).

20. A cervical collar according to claim 15, wherein the electronic monitoring device has a built in alarm.

21. A cervical collar according to claim 15, wherein intervals between alarms can be programmed in the electronic monitoring device.

22. A method for treating neck injuries, including whiplash injuries, wherein
a cervical collar according to claim 1 is applied around a damaged neck of a patient; and
the length of the supportive connection means is adjusted to the appropriate length and angle to assure a proper fit for the centre of rotation and rotational plane of the patient's neck; and
in early stages of an healing process or during instability, circular horizontal rotation between the lower neck support piece in relation to the neck support piece is restricted with respect to each other by adjustable regulating means; and
when prescribed or needed the patient performs rotation exercises by rotating the head and neck to the left and/or right to an allowed degree of rotation around the rotation centre and rotation plane.

23. A method according to claim 19, wherein the performance of rotation exercises is monitored by means of an electronic monitoring device.

24. A cervical collar according to claim 1, wherein the attachment between the shoulder and back support and the lower neck support piece is provided by at least three supportive connecting means.
